(19) United States
(12) Patent Application Publication
(10) Pub. No.: US 2011/0312986 A1
(43) Pub. Date: Dec. 22, 2011

(54) Composition and Method for Treating Overactive Bladder

(75) Inventor: Michael G. Oefelein, Tustin, CA (US)

(73) Assignee: ALLERGAN, INC., Irvine, CA (US)

(21) Appl. No.: 13/162,424

(22) Filed: Jun. 16, 2011

Related U.S. Application Data
(60) Provisional application No. 61/355,486, filed on Jun. 16, 2010.
(60) Provisional application No. 61/358,357, filed on Jun. 24, 2010.

(51) Int. Cl. A61K 31/438; A61P 13/10

(52) U.S. Cl. 514/278

(57) ABSTRACT

The present specification discloses compounds, compositions including such compounds, kits including such compounds and compositions, and methods of treating an individual suffering from overactive bladder and/or diabetes by administering such compounds or compositions to an individual in need thereof.

This patent application claims priority pursuant to 35 U.S.C. §119(e) to U.S. Provisional Patent Application Ser. No. 61/355,486 filed Jun. 16, 2010, and U.S. Provisional Patent Application Ser. No. 61/358,357 filed Jun. 24, 2010; each of which is hereby incorporated by reference in its entirety.

Overactive bladder is a chronic condition characterized by the lower urinary tract symptoms of urinary urgency, with or without urge incontinence, usually with urinary frequency and nocturia. Overactive bladder is the most common cause of urinary incontinence or loss of bladder control in adults and affects approximately 33 million, or about 17%, of adults in the U.S.A. Although the prevalence among men and women in the U.S.A. is similar (16.0% vs. 16.9%, respectively), the severity and nature of symptom expression does differ, with women demonstrating a higher incidence of urge incontinence. There is also a marked increase in prevalence with increasing age. Overactive bladder has a significant impact on the health-related quality of life, mental health, and quality of sleep of affected individuals, whether or not they display the symptom of urge incontinence. The economic burden of overactive bladder is also significant, estimated at approximately $12 billion per annum in the U.S.A. alone. Overactive bladder is distinct from stress urinary incontinence, but when they occur together is usually known as mixed incontinence.

The etiology of overactive bladder is unclear, and indeed there may be multiple possible causes. Symptoms of overactive bladder are usually attributed to detrusor muscle overactivity, a pattern of bladder muscle contraction observed during urodynamics, during the bladder filling/urine storage phase, and may be neurogenic, myogenic, or idiopathic in nature. Treatments for overactive bladder, are usually synonymous with treatments for detrusor overactivity and include lifestyle modification (fluid restriction, avoidance of caffeine), bladder retraining, antimuscarinic drugs (Darifenacin, Hyoscyamine, Oxybutynin, Tolterodine, Solifenacin, Trospium), and various devices (Urgent PC Neuromodulation System, InterStim). Intravesical botulinum toxin A is also used in some intractable cases, although not with formal FDA approval.

Although a wide variety of treatments are currently available, there is still a need to develop better treatments. The present specification discloses that co-administering Trospium with Metformin results in unique pharmacokinetic properties of Trospium that reduces side effects.

### SUMMARY

Aspects of the present specification disclose a composition comprising a Trospium and a Metformin. This composition provides enhanced efficacy of treating urination frequency and urgency with lower side effects compared to treating overactive bladder with Trospium alone.

Other aspects of the present specification disclose a method of treating overactive bladder comprising administration of a composition comprising a Trospium and a Metformin.

Other aspects of the present specification disclose a method of treating overactive bladder comprising administration of a composition comprising a Trospium and a composition comprising a Metformin.

Yet other aspects of the present specification disclose a pharmaceutical kit comprising a composition including comprising a therapeutically effective amount of a Trospium and a therapeutically effective amount of a Metformin and instructions on how to administer the composition to an individual suffering from an overactive bladder disorder and/or diabetes.

Still other aspects of the present specification disclose a method of selecting a treatment for an individual suffering from an overactive bladder disorder, wherein a compound or composition disclosed herein is selected to provide an effective treatment from the overactive bladder disorder without the compound or composition entering the central nervous system.

### BRIEF DESCRIPTION OF THE DRAWINGS

FIG. 1 shows a graph of steady-state plasma concentration-time profiles of Trospium chloride following oral administration of 500 mg Metformin twice daily alone or in combination with 60 mg Trospium chloride administered once daily in a two-period cross-over study design (mean+/−SD).

FIG. 2 shows a graph of steady-state plasma concentration-time profiles of Metformin following oral administration of 500 mg Metformin twice daily alone or in combination with 60 mg Trospium chloride administered once daily in a two-period cross-over study design (mean+/−SD).

### DETAILED DESCRIPTION

Trospium, commercially available as SANCTURA XR® (Trospium chloride), belongs to a drug class of anticholinergic agents and is a non-selective muscarinic receptor antagonist. Trospium is used for the treatment of overactive bladder (OAB) and works by relaxing smooth muscle tissue found in the bladder, thus decreasing bladder spasms that are thought to be a cause of OAB, see, e.g., U.S. Pat. No. 7,410,978, which is hereby incorporated by reference in its entirety.

Trospium is a cationic quaternary amine which is not extensively metabolized via CYP450, and it is thought to be metabolized by non-inducible ester hydrolysis. Its metabolite, azoniaspironortropanol, is pharmacologically inactive. The majority of the absorbed dose of Trospium (60%) is excreted unchanged in the urine through tubular secretion and glomerular filtration. Based on in vitro metabolism data, no clinically relevant metabolic (e.g., CYP450) drug-drug interactions are anticipated with Trospium. However, there may be a competition for elimination with other compounds that are also renally eliminated. One such potential drug-drug interaction is between Digoxin, a Pglycoprotein mediated mechanism of renal elimination and Trospium.

Another possible renal-based drug-drug interaction is between Metformin and Trospium. Metformin, commercially available as GLUCOPHAGE® (Metformin hydrochloride), is an antihyperglycemic drug in the biguanide class. It is recommended as first-line drug of choice for the treatment of type 2 diabetes, in particular, in overweight and obese people and those with normal kidney function see, e.g., U.S. Pat. No. 6,660,300 and U.S. Pat. No. 6,667,054, each of which are incorporated by reference in its entirety. Evidence is also mounting for its efficacy in gestational diabetes, although safety concerns still preclude its widespread use in this setting. It is also used in the treatment of polycystic ovary syndrome, and has been investigated for other diseases where insulin resistance may be an important factor.

Diabetes is a condition in which a person has high blood sugar, either because the body doesn't produce enough insulin, or because cells don't respond to the insulin that is produced. All forms of diabetes have been treatable since insulin became available in 1921, and type 2 diabetes may be controlled with medications. Diabetes without proper treatments can cause many complications. Acute complications include hypoglycemia, diabetic ketoacidosis, or nonketotic hyperosmolar coma. Serious long-term complications include cardiovascular disease, chronic renal failure, retinal damage. Adequate treatment of diabetes is thus important, as well as blood pressure control and lifestyle factors such as smoking cessation and maintaining a healthy body weight. As of 2000 at least 171 million people worldwide suffer from diabetes, or 2.8% of the population.

Metformin is both filtered and secreted by the kidney. The proximal tubular secretion of Metformin occurs via the organic cation transport system. Drug-drug interactions have been reported between Metformin, Procanamide and Cimetidine, and this interaction suggests that cationic drugs that are eliminated by renal tubular secretion (e.g., Trospium) theoretically have the potential for interaction with Metformin by competing for common renal tubular transport systems. Therefore, the concurrent administration of Metformin and Trospium may potentially affect the excretion of each compound, and if the renal clearance of these compounds is altered, there may be a corresponding effect on the concentration and the clinical response.

Urinary frequency and urgency are also a classic symptoms of diabetes mellitus. In addition, diabetes is a condition in which a person has high blood sugar. The high blood sugar causes osmotic dieresis thus decreasing reabsorption of water and consequently increasing urine output. The administration of Metformin suppresses hepatic glucose production, increases insulin sensitivity, enhances peripheral glucose uptake (by phosphorylating GLUT-4 enhancer factor), increases fatty acid oxidation, and decreases absorption of glucose from the gastrointestinal tract. Therefore, an individual who suffers from diabetes may also suffer from an overactive bladder disorder.

Thus, Trospium address overactive bladder by treating the spasms associated with this disorder by relaxing the smooth muscles of the bladder. Metformin treats overactive bladder associated with diabetes by relieving osmotic dieresis and reducing urine output. Building on these findings, it is believed that combining the use of Trospium and Metformin will treat multiple causes of the same symptoms, urinary frequency and urgency of overactive bladder, thereby providing a synergistic therapeutic value.

Aspects on the present specification disclose, in part, a Trospium. As used herein the term “Trospium” refers to the compound 3-(2-hydroxy-2,2-diphenylacetoxy) spiro[bicyclo[3.2.1]octane-8,1′-pyrrolidin]-1′-ium chloride and any pharmaceutically-acceptable salt thereof. The chemical formula for Trospium is given in formula I.

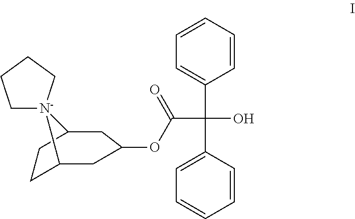

One particular Trospium salt is Trospium chloride which is commercially marketed under the name SANTURA XR® (Allergan, Inc., Irvine, Calif.).

Aspects on the present specification disclose, in part, a Metformin. As used herein the term “Metformin” refers to the compound N,N-dimethylimidodicarbonimidic diamide, or, equivalently, N,N-dimethylbiguanide and any pharmaceutically-acceptable salt thereof. The chemical formula for Metformin is given in formula II.

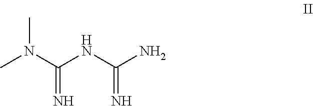

One particular Metformin salt is Metformin hydrochloride which is commercially marketed under the name GLUCOPHAGE® (Bristol-Myers Squibb Co., New York, N.Y.).

Aspects of the present specification disclose, in part, a composition comprising a Trospium, a composition comprising a Metformin, or a composition comprising a Trospium and a Metformin. In aspects of this embodiment, the amount of a Trospium and/or a Metformin is a therapeutically effective amount.

The combination product may be a blended form of the Trospium and Metformin. The combination product may be administered orally or through other means commonly known for the administration of drugs. The combination product may be in the form of a capsule and administered once daily therefore simultaneously administering the drug, or the combination product may be administered multiple times daily using mulitople capsules. Pills, tablets, and other administration methods commonly known to one skilled in the art may be used.

A compound or a composition disclosed herein is generally administered to an individual as a pharmaceutical composition. Pharmaceutical compositions may be prepared by combining a therapeutically effective amount of at least one compound according to the present specification, or a pharmaceutically acceptable acid addition salt thereof, as an active ingredient, with conventional acceptable pharmaceutical excipients, and by preparation of unit dosage forms suitable for topical ocular use. The therapeutically effective amount typically is between about 5 mg and about 100 mg for Trospium and about 50 mg to about 1,000 mg for Metformin. As used herein, the term “pharmaceutical composition” and refers to a therapeutically effective concentration of an active compound, such as, e.g., any of the compounds disclosed herein. Preferably, the pharmaceutical composition does not produce an adverse, allergic, or other untoward or unwanted reaction when administered to an individual. A pharmaceutical composition disclosed herein is useful for medical and veterinary applications. A pharmaceutical composition may be administered to an individual alone, or in combination with other supplementary active compounds, agents, drugs or hormones. The pharmaceutical compositions may be manufactured using any of a variety of processes, including, without limitation, conventional mixing, dissolving, granulating, dragee-making, levigating, emulsifying, encapsulating, entrapping, and lyophilizing. The pharmaceutical composition can take any of a variety of forms including, without limitation, a sterile solution, suspension, emulsion, lyophilizate, tablet, pill, pellet, capsule, powder, syrup, elixir, or any other dosage form suitable for administration. In one embodiment, the composition is in a dosage form that can be administered orally.

The compounds disclosed herein may also be incorporated into a drug delivery platform in order to achieve a controlled compound release profile over time. Controlled release is a mechanism used in pill tablets or capsules to dissolve slowly and release a drug over time. The advantages of controlled-release tablets or capsules are that they can often be taken less frequently than instant-release formulations of the same drug, and that they keep steadier levels of the drug in the bloodstream.

Most controlled-release drugs are formulated so that the active ingredient is embedded in a matrix of insoluble substance(s) such that the dissolving drug must find its way out through the holes in the matrix. Some drugs are enclosed in polymer-based tablets with a laser-drilled hole on one side and a porous membrane on the other side. Stomach acids push through the porous membrane, thereby pushing the drug out through the laser-drilled hole. In time, the entire drug dose releases into the system while the polymer container remains intact, to be later excreted through normal digestion. In some sustained release formulations, the drug dissolves into the matrix, and the matrix physically swells to form a gel, allowing the drug to exit through the gel's outer surface.

Such a controlled release formulation comprises a compound disclosed herein dispersed within a polymer matrix, typically a biodegradable, bioerodible, and/or bioresorbable polymer matrix. As used herein, the term “polymer” refers to synthetic homo- or copolymers, naturally occurring homo- or copolymers, as well as synthetic modifications or derivatives thereof having a linear, branched or star structure. Copolymers can be arranged in any form, such as, e.g., random, block, segmented, tapered blocks, graft, or triblock. Polymers are generally condensation polymers. Polymers can be further modified to enhance their mechanical or degradation properties by introducing cross-linking agents or changing the hydrophobicity of the side residues. If crosslinked, polymers are usually less than 5% crosslinked, usually less than 1% crosslinked.

Suitable polymers include, without limitation, alginates, aliphatic polyesters, polyalkylene oxalates, polyamides, polyamidoesters, polyanhydrides, polycarbonates, polyesters, polyethylene glycol, polyhydroxyaliphatic carboxylic acids, polyorthoesters, polyoxaesters, polypeptides, polyphosphazenes, polysaccharides, and polyurethanes. The polymer usually comprises at least about 10% (w/w), at least about 20% (w/w), at least about 30% (w/w), at least about 40% (w/w), at least about 50% (w/w), at least about 60% (w/w), at least about 70% (w/w), at least about 80% (w/w), or at least about 90% (w/w) of the drug delivery platform. Examples of biodegradable, bioerodible, and/or bioresorbable polymers and methods useful to make a controlled release formulation are described in, e.g., Drost, et. al., Controlled Release Formulation, U.S. Pat. No. 4,756,911; Smith, et. al., Sustained Release Drug Delivery Devices, U.S. Pat. No. 5,378,475; Wong and Kochinke, Formulation for Controlled Release of Drugs by Combining Hyrophilic and Hydrophobic Agents, U.S. Pat. No. 7,048,946; Hughes, et. Al., Compositions and Methods for Localized Therapy of the Eye, U.S. Patent Publication 2005/0181017; Hughes, Hypotensive Lipid-Containing Biodegradable Intraocular Implants and Related Methods, U.S. Patent Publication 2005/0244464; Altman, et al., Silk Fibroin Hydrogels and Uses Thereof, U.S. patent application Ser. No. 12/764,039, filed on Apr. 20, 2010; each of which is incorporated by reference in its entirety.

In aspects of this embodiment, a polymer composing the matrix is a polypeptide such as, e.g., silk fibroin, keratin, or collagen. In other aspects of this embodiment, a polymer composing the matrix is a polysaccharide such as, e.g., cellulose, agarose, elastin, chitosan, chitin, or a glycosaminoglycan like chondroitin sulfate, dermatan sulfate, keratan sulfate, or hyaluronic acid. In yet other aspects of this embodiment, a polymer composing the matrix is a polyester such as, e.g., D-lactic acid, L-lactic acid, racemic lactic acid, glycolic acid, caprolactone, and combinations thereof.

One of ordinary skill in the art appreciates that the selection of a suitable polymer for forming a suitable disclosed controlled release formulation depends on several factors. The more relevant factors in the selection of the appropriate polymer(s), include, without limitation, compatibility of polymer with drug, desired release kinetics of drug, desired biodegradation kinetics of formulation at implantation site, desired bioerodible kinetics of formulation at implantation site, desired bioresorbable kinetics of formulation at implantation site, in vivo mechanical performance of formulation, processing temperatures, biocompatibility of formulation, and patient tolerance. Other relevant factors that, to some extent, dictate the in vitro and in vivo behavior of the polymer include the chemical composition, spatial distribution of the constituents, the molecular weight of the polymer and the degree of crystallinity.

A controlled release formulation includes both a sustained release drug delivery platform and an extended release drug delivery platform. As used herein, the term “sustained release” refers to the release of a compound disclosed herein over a period of about seven days or more. As used herein, the term “extended release” refers to the release of a compound disclosed herein over a period of time of less than about seven days.

In aspects of this embodiment, a sustained release drug delivery platform releases a compound disclosed herein with substantially first order release kinetics over a period of, e.g., about 7 days after administration, about 15 days after administration, about 30 days after administration, about 45 days after administration, about 60 days after administration, about 75 days after administration, or about 90 days after administration. In other aspects of this embodiment, a sustained release drug delivery platform releases a compound disclosed herein with substantially first order release kinetics over a period of, e.g., at least 7 days after administration, at least 15 days after administration, at least 30 days after administration, at least 45 days after administration, at least 60 days after administration, at least 75 days after administration, or at least 90 days after administration.

In aspects of this embodiment, a drug delivery platform releases a compound disclosed herein with substantially first order release kinetics over a period of, e.g., about 1 day after administration, about 2 days after administration, about 3 days after administration, about 4 days after administration, about 5 days after administration, or about 6 days after administration. In other aspects of this embodiment, a drug delivery platform releases a compound disclosed herein with substantially first order release kinetics over a period of, e.g., at most 1 day after administration, at most 2 days after administration, at most 3 days after administration, at most 4 days after administration, at most 5 days after administration, or at most 6 days after administration.

Delayed release is a mechanism by which a drug can be released at a time later than that immediately following its administration into the human body. Oral medicines that do not immediately disintegrate and release the active ingredient(s) into the body are known as delayed release medicines. An example is enteric coated oral medications, which dissolve in the intestines rather than the stomach.

A pharmaceutical composition disclosed herein can optionally include a pharmaceutically acceptable carrier that facilitates processing of an active compound into pharmaceutically acceptable compositions. As used herein, the term “pharmacologically acceptable carrier” is synonymous with “pharmacological carrier” and refers to any carrier that has substantially no long term or permanent detrimental effect when administered and encompasses terms such as “pharmacologically acceptable vehicle, stabilizer, diluent, additive, auxiliary, or excipient.” Such a carrier generally is mixed with an active compound or permitted to dilute or enclose the active compound and can be a solid, semi-solid, or liquid agent. It is understood that the active compounds can be soluble or can be delivered as a suspension in the desired carrier or diluent. Any of a variety of pharmaceutically acceptable carriers can be used including, without limitation, aqueous media such as, e.g., water, saline, glycine, hyaluronic acid and the like; solid carriers such as, e.g., starch, magnesium stearate, mannitol, sodium saccharin, talcum, cellulose, glucose, sucrose, lactose, trehalose, magnesium carbonate, and the like; solvents; dispersion media; coatings; antibacterial and antifungal agents; isotonic and absorption delaying agents; or any other inactive ingredient. Selection of a pharmacologically acceptable carrier can depend on the mode of administration. Except insofar as any pharmacologically acceptable carrier is incompatible with the active compound, its use in pharmaceutically acceptable compositions is contemplated. Non-limiting examples of specific uses of such pharmaceutical carriers can be found in Pharmaceutical Dosage Forms and Drug Delivery Systems (Howard C. Ansel et al., eds., Lippincott Williams & Wilkins Publishers, 7th ed. 1999); Remington: The Science and Practice of Pharmacy (Alfonso R. Gennaro ed., Lippincott, Williams & Wilkins, 20th ed. 2000); Goodman & Gilman's The Pharmacological Basis of Therapeutics (Joel G. Hardman et al., eds., McGraw-Hill Professional, 10th ed. 2001); and Handbook of Pharmaceutical Excipients (Raymond C. Rowe et al., APhA Publications, 4th edition 2003). These protocols are routine and any modifications are well within the scope of one skilled in the art and from the teaching herein.

A pharmaceutical composition disclosed herein can optionally include, without limitation, other pharmaceutically acceptable components (or pharmaceutical components), including, without limitation, buffers, preservatives, tonicity adjusters, salts, antioxidants, osmolality adjusting agents, physiological substances, pharmacological substances, bulking agents, emulsifying agents, wetting agents, sweetening or flavoring agents, and the like. Various buffers and means for adjusting pH can be used to prepare a pharmaceutical composition disclosed herein, provided that the resulting preparation is pharmaceutically acceptable. Such buffers include, without limitation, acetate buffers, borate buffers, citrate buffers, phosphate buffers, neutral buffered saline, and phosphate buffered saline. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole, and butylated hydroxytoluene. Useful preservatives include, without limitation, benzalkonium chloride, chlorobutanol, thimerosal, phenylmercuric acetate, phenylmercuric nitrate, a stabilized oxy chloro composition, such as, e.g., sodium chlorite and chelants, such as, e.g., DTPA or DTPA-bisamide, calcium DTPA, and CaNaDTPA-bisamide. Tonicity adjustors useful in a pharmaceutical composition include, without limitation, salts such as, e.g., sodium chloride, potassium chloride, mannitol or glycerin and other pharmaceutically acceptable tonicity adjustor. The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. It is understood that these and other substances known in the art of pharmacology can be included in a pharmaceutical composition useful in the invention.

Aspects of the present specification disclose, in part, treating an individual suffering from an overactive bladder disorder. As used herein, the term “treating,” refers to reducing or eliminating in an individual a clinical symptom of an overactive bladder disorder; or delaying or preventing in an individual the onset of a clinical symptom of an overactive bladder disorder. For example, the term “treating” can mean reducing a symptom of a condition characterized by an overactive bladder disorder by, e.g., at least 20%, at least 30%, at least 40%, at least 50%, at least 60%, at least 70%, at least 80%, at least 90% or at least 100%. The effectiveness of a compound or composition disclosed herein in treating a condition characterized by an overactive bladder disorder can be determined by observing one or more clinical symptoms, and/or physiological indicators associated with the condition. An improvement in an overactive bladder disorder also can be indicated by a reduced need for a concurrent therapy. Those of skill in the art will know the appropriate symptoms or indicators associated with specific overactive bladder disorder and will know how to determine if an individual is a candidate for treatment with a compound or composition disclosed herein.

A composition or compound is administered to an individual. An individual is typically a human being. Typically, any individual who is a candidate for a conventional overactive bladder treatment is a candidate for an overactive bladder disorder treatment disclosed herein. Pre-operative evaluation typically includes routine history and physical examination in addition to thorough informed consent disclosing all relevant risks and benefits of the procedure.

The compounds, compositions and methods disclosed herein are useful in treating an overactive bladder disorder. An Overactive bladder is increased urinary urgency, with or without urge urinary incontinence, usually with frequency and nocturia. The individual may report symptoms of urinary urgency (the sudden, intense desire to urinate immediately), urinary frequency (the need to urinate more times than is normal), enuresis (any involuntary loss of urine), polyuria, nocturia, and/or urinary incontinence. Thus, overactive bladder describes a bladder that contracts more often than it should, so that a person feels the need to urinate more frequently and/or urgently than necessary and is characterized by uncontrolled, frequent expulsion of urine from the bladder. An overactive bladder usually, but not always, causes urinary incontinence. Individuals with overactive bladder may go to the bathroom very often, e.g., every two hours during the day and night, and may even wet the bed. Often, a strong urge to void is experienced when only a small amount of urine is in the bladder. There may be reduced bladder capacity and incomplete emptying of urine. An overactive bladder can be caused by interruptions in the nerve pathways to the bladder occurring above the sacrum. For example, spastic bladder may be caused by an inability of the detrusor muscle of the bladder to inhibit emptying contractions until a reasonable amount of urine has accumulated. As such, overactive bladder is often associated with detrusor overactivity, a pattern of bladder muscle contraction observed during urodynamics. Overactive bladder can also be caused by urinary tract infection, outflow obstruction and stress incontinence. Sometimes no cause is found, and such idiopathic cases may be due to anxiety or aging. Symptoms include the need to urinate may times throughout the day and night, the sensation of having to urinate immediately, and/or the sudden leakage of urine from the bladder.

Diseases extrinsic to the bladder may also cause the symptoms of overactive bladder. In the male patient, the extrinsic disorder most often responsible for overactive bladder is bladder outlet obstruction (BOO). Disorders extrinsic to the bladder in the female patient include urethral diverticulum, retroverted uterus, pelvic prolapse (including cystocele), gravid uterus, and loss or reduction of estrogen. Disorders extrinsic to the bladder common to both men and woman include pelvic mass, physiologic nocturnal diuresis, and polyuria caused by factors such as excessive fluid intake, diuretic use, or diabetes. Neuromuscular disorders may also account for the overactive bladder. Neurogenic disorders resulting from nerve damage to sensory nerves can also cause overactive bladder, including, without limitation, Parkinson disease, multiple sclerosis, spina bifida, cervical stenosis, spinal cord injury, diabetic neuropathy, pelvic surgery, or invertebral disc herniation, hydrocephalus, stroke, spinal cord injuries and lesions of the spinal cord or brain. Bladder aging may also account for these symptoms. A patient history of pelvic trauma, pelvic radiation, or bladder, prostate, or urethral surgery should also be considered when seeking to determine the etiology of the overactive bladder.

Thus in embodiment, an individual suffering from overactive bladder is treated with a composition comprising a therapeutically effective amount of a Trospium and a therapeutically effective amount of a Metformin wherein such administration reduces a symptom associated with the overactive bladder. In an aspect of this embodiment, an individual suffering from overactive bladder is treated with a composition comprising a therapeutically effective amount of a modified Clostridial toxin where such administration reduces incontinence. In an aspect of this embodiment, an individual suffering from overactive bladder is treated with a composition comprising a therapeutically effective amount of a Trospium and a therapeutically effective amount of a Metformin wherein such administration reduces urinary frequency. In another aspect of this embodiment, an individual suffering from overactive bladder is treated with a composition comprising a therapeutically effective amount of a Trospium and a therapeutically effective amount of a Metformin wherein such administration reduces urinary urgency. In another aspect of this embodiment, an individual suffering from overactive bladder is treated with a composition comprising a therapeutically effective amount of a Trospium and a therapeutically effective amount of a Metformin wherein such administration reduces enuresis. In another aspect of this embodiment, an individual suffering from overactive bladder is treated with a composition comprising a therapeutically effective amount of a Trospium and a therapeutically effective amount of a Metformin wherein such administration reduces polyuria. In yet another aspect of this embodiment, an individual suffering from overactive bladder is treated with a composition comprising a therapeutically effective amount of a Trospium and a therapeutically effective amount of a Metformin wherein such administration reduces nocturia. In yet another aspect of this embodiment, an individual suffering from overactive bladder is treated with a composition comprising a therapeutically effective amount of a Trospium and a therapeutically effective amount of a Metformin wherein such administration reduces urinary incontinence.

In another aspects of this embodiment, a therapeutically effective amount of a Trospium is administered to an individual before administration of a therapeutically effective amount of a Metformin. In yet another aspects of this embodiment, a therapeutically effective amount of a Metformin is administered to an individual before administration of a therapeutically effective amount of a Trospium. In still another aspects of this embodiment, a therapeutically effective amount of a Metformin and a therapeutically effective amount of a Trospium are administered simultaneously to an individual. When administered simultaneously, the Trospium and Metformin may be administered as separate compositions or may be administered as a single composition comprising both Trospium and Metformin.

The amount of a compound used with any of the methods disclosed herein will typically be a therapeutically effective amount. As used herein, the term “therapeutically effective amount” is synonymous with “therapeutically effective dose” and refers to the amount of compound that will elicit the biological or clinical response being sought by the practitioner in an individual in need thereof. As a non-limiting example, an effective amount is an amount sufficient to reduce a symptom of overactive bladder like urinary urgency, urinary frequency, enuresis, polyuria, nocturia, and/or urinary incontinence. The appropriate effective amount to be administered for a particular application of the disclosed methods can be determined by those skilled in the art, using the guidance provided herein. For example, an effective amount can be extrapolated from in vitro and in vivo assays as described in the present specification. One skilled in the art will recognize that the condition of the individual can be monitored throughout the course of therapy and that the effective amount of a compound or composition disclosed herein that is administered can be adjusted accordingly.

In aspects of this embodiment, a therapeutically effective amount of a composition comprising a compound or compounds disclosed herein reduces a symptom associated with an overactive bladder disorder and/or diabetes by, e.g., at least 10%, at least 20%, at least 30%, at least 40%, at least 50%, at least 60%, at least 70%, at least 80%, at least 90% or at least 100%. In other aspects of this embodiment, a therapeutically effective amount of a composition comprising a compound or compounds disclosed herein reduces a symptom associated with an overactive bladder disorder and/or diabetes by, e.g., at most 10%, at most 20%, at most 30%, at most 40%, at most 50%, at most 60%, at most 70%, at most 80%, at most 90% or at most 100%. In yet other aspects of this embodiment, a therapeutically effective amount of a composition comprising a compound or compounds disclosed herein reduces a symptom associated with an overactive bladder disorder and/or diabetes by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%. In still other aspects of this embodiment, a therapeutically effective amount of a compound or compounds disclosed herein is the dosage sufficient to reduces a symptom associated with an overactive bladder disorder and/or diabetes for, e.g., at least one week, at least one month, at least two months, at least three months, at least four months, at least five months, at least six months, at least seven months, at least eight months, at least nine months, at least ten months, at least eleven months, or at least twelve months.

In aspects of this embodiment, the amount of a Trospium administered to an individual is, e.g., about 5 mg, about 10 mg, about 20 mg, about 30 mg, about 40 mg, about 50 mg, about 60 mg, about 70 mg, about 80 mg, about 90 mg, or about 100 mg; and the amount of a Metformin administered to an individual is, e.g., about 50 mg, about 100 mg, about 200 mg, about 300 mg, about 400 mg, about 500 mg, about 600 mg, about 700 mg, about 800 mg, about 900 mg, or about 1,000 mg.

In other aspects of this embodiment, the amount of a Trospium administered to an individual is, e.g., at least 5 mg, at least 10 mg, at least 20 mg, at least 30 mg, at least 40 mg, at least 50 mg, at least 60 mg, at least 70 mg, at least 80 mg, at least 90 mg, or at least 100 mg; and the amount of a Metformin administered to an individual is, e.g., at least 50 mg, at least 100 mg, at least 200 mg, at least 300 mg, at least 400 mg, at least 500 mg, at least 600 mg, at least 700 mg, at least 800 mg, at least 900 mg, or at least 1,000 mg.

In yet other aspects of this embodiment, the amount of a Trospium administered to an individual is, e.g., at most 5 mg, at most 10 mg, at most 20 mg, at most 30 mg, at most 40 mg, at most 50 mg, at most 60 mg, at most 70 mg, at most 80 mg, at most 90 mg, or at most 100 mg; and the amount of a Metformin administered to an individual is, e.g., at most 50 mg, at most 100 mg, at most 200 mg, at most 300 mg, at most 400 mg, at most 500 mg, at most 600 mg, at most 700 mg, at most 800 mg, at most 900 mg, or at most 1,000 mg.

In another aspect of this embodiment, the amount of a Trospium administered to an individual is between, e.g., about 5 mg to about 100 mg and the amount of a Metformin administered to an individual is, e.g., about 50 mg to about 1,000 mg. In yet another aspect of this embodiment, the amount of a Trospium administered to an individual is between, e.g., about 5 mg to about 50 mg and the amount of a Metformin administered to an individual is, e.g., about 50 mg to about 500 mg. In still another aspect of this embodiment, the amount of a Trospium administered to an individual is between, e.g., about 5 mg to about 40 mg and the amount of a Metformin administered to an individual is, e.g., about 50 mg to about 400 mg. In another aspect of this embodiment, the amount of a Trospium administered to an individual is between, e.g., about 5 mg to about 30 mg and the amount of a Metformin administered to an individual is, e.g., about 50 mg to about 300 mg. In yet another aspect of this embodiment, the amount of a Trospium administered to an individual is between, e.g., about 5 mg to about 25 mg and the amount of a Metformin administered to an individual is, e.g., about 50 mg to about 250 mg. In still another aspect of this embodiment, the amount of a Trospium administered to an individual is between, e.g., about 5 mg to about 20 mg and the amount of a Metformin administered to an individual is, e.g., about 50 mg to about 200 mg.

In another aspect of this embodiment, the amount of a Trospium administered to an individual is about 50 mg and the amount of a Metformin administered to an individual is about 500 mg. In another aspect of this embodiment, the amount of a Trospium administered to an individual is about 40 mg and the amount of a Metformin administered to an individual is about 400 mg. In another aspect of this embodiment, the amount of a Trospium administered to an individual is about 30 mg and the amount of a Metformin administered to an individual is about 300 mg. In another aspect of this embodiment, the amount of a Trospium administered to an individual is about 25 mg and the amount of a Metformin administered to an individual is about 250 mg. In another aspect of this embodiment, the amount of a Trospium administered to an individual is about 20 mg and the amount of a Metformin administered to an individual is about 200 mg.

The route of administration of a compound or composition administered to an individual will typically be determined based on the clinical effect desired by the individual and/or physician. In one embodiment, a composition may be suitable for oral administration that contains at least two active ingredients, one of these being Trospium and the other Metformin for effective treatment of urinary frequency and urgency. In another embodiment, a method for treating diabetes and overactive bladder may be performed by oral administering a composition containing at least two active ingredients, one of those being Trospium and the other Metformin.

Aspects of the present specification disclose, in part, a pharmaceutical kit. In one embodiment, the kit includes a pharmaceutical composition comprising a therapeutically effective amount of a Trospium, and a pharmaceutical composition comprising a therapeutically effective amount of a Metformin. In another embodiment, the kit includes a pharmaceutical composition comprising a therapeutically effective amount of a Trospium and a therapeutically effective amount of a Metformin. A pharmaceutical kit optionally includes instructions on how to safely administer the compositions to an individual suffering from overactive bladder and/or diabetes.

### EXAMPLES

The following non-limiting examples are provided for illustrative purposes only in order to facilitate a more complete understanding of representative embodiments now contemplated. These examples should not be construed to limit any of the embodiments described in the present specification, including those pertaining to the compounds, compositions and methods of treating overactive bladder and/or diabetes disclosed herein.

### Example 1

A single-center, single-blind, randomized, placebo-controlled, parallel group study was conducted to evaluate whether Trospium permeates across the human blood-brain barrier and whether Trospium could cause cognitive impairment.

A total of 12 healthy subjects with symptoms of overactive bladder were enrolled in this study. All subjects were 65 years of age or older. All subjects were between 65 and 75 years of age with a mean age of 67 years. Seven subjects were female and five subjects were male. All 12 subjects completed the study.

Each subject received a daily dose of 60 mg Trospium over a 10 day period. For plasma sampling at each pharmacokinetics time point, about 10 mL of blood was collected at on Day 10 at 0 hr, 2 hr, 5 hr, 7 hr, 12 hr, and 24 hr post dose. Each sample was drawn into a green-top sodium heparin tube, and inverted at least 10 times to insure adequate mixing of blood and anticoagulant. The green-top tube was placed in an ice bath for a minimum of 5 minutes before being centrifuged for 10 minutes at approximately 2,000 g at 4° C. Immediately after centrifugation, the plasma samples were divided by transferring into 2 separate duplicate labeled polypropylene cryovials (minimum of 1 mL plasma in each). Plasma samples were kept frozen at all times (−20° C. or below) until bioanalysis.

For cerebrospinal fluid sampling at each pharmacokinetics time point, about 2.5 mL of cerebral spinal fluid was collected on Day 10 at 0 hr, 2 hr, 5 hr, 7 hr, 12 hr, and 24 hr post dose. Immediately after collection, the cerebrospinal fluid samples were divided by transferring into 2 separate duplicate labeled polypropylene cryovials (minimum of 1 mL plasma in each) and gently mixing to avoid gradient effects. Cerebrospinal fluid samples were kept frozen at all times (−20° C. or below) until bioanalysis.

The concentrations of Trospium in plasma and cerebral spinal fluid were measured using a validated liquid chromatography tandem mass spectrometry method (LC-MS/MS). All bioanalytical methods showed acceptable precision and accuracy for the quality control samples, and all plasma and urine samples were analyzed within pre-determined stability window.

For neuropsychology testing, each subject underwent the Hopkins Verbal Learning Test-Revised (HVLT-R) and the Brief Visuospatial Memory Test-Revised (BVMT-R) on Days 0, 9, and 10. Pre-dose (Day 0) and post-dose (Day 10) results on the neuropsychology tests were compared using a reliable change index to assess a change in learning of memory.

Peak plasma concentration of Trospium (Cₘₐₓ) in this study was 925 pg/mL and occurred at 5 hr post-dosing. The steady state area under the plasma concentration-time curve within dosing interval was calculated by the linear trapezoidal rule, and an AUC₀₋₂₄ value of 18,500 ng·hr/mL was obtained. Despite measureable peal plasma steady state values, mean Trospium levels in the cerebrospinal fluid of all subjects was undetectable (<40 pg/mL) on Day 10 at steady state peak plasma concentrations at 5 hours post dose. In fact, a total of 72 cerebrospinal fluid samples were evaluated and all fell below the detectable range of 40 pg/mL for Trospium. These results indicate that Trospium does not cross the blood-brain barrier.

TABLE 1
Trospium Concentrations in Plasma and Cerebrospinal Fluid

| | Time | | |
|---|---|---|---|
| Sample | 0 hr | 5 hr | 24 hr |
| Plasma | 522 pg/mL | 925 pg/mL | 649 pg/mL |
| Cerebrospinal Fluid | <40 pg/mL | <40 pg/mL | <40 pg/mL |

Repeat neuropsychology testing revealed no meaningful net drug effect on cognition, although one subject experienced significant deterioration on the total recall portion of both tests. These results indicate that Trospium does not cause cognitive impairment or memory loss.

### Example 2

Both Trospium and Metformin is both filtered and secreted by the kidney. To investigate whether a potential adverse renal-based drug-drug interaction occurs between these two compounds, a single-center, randomized, open-label, two-period, cross-over study was conducted to assess the pharmacokinetics, pharmacodynamics, safety and tolerability of co-administration of Trospium and Metformin under steady state conditions in healthy subjects.

A total of 44 healthy subjects were enrolled in this study. All subjects were between 18 and 44 years of age with a mean age of 31 years (Table 1). The subjects ranged in height from 153 to 180 cm with a mean of 165 cm (Table 2). Their weights ranged from 55.3 to 87.9 kg with a mean of 69.5 kg (Table 2). Among the 44 subjects enrolled in the study, 3 subjects were Caucasian and 41 subjects were Hispanic. Nineteen subjects were female and 25 subjects were male. A total of 43 subjects completed the study and one subject was discontinued by the investigator due to flu-like symptoms.

TABLE 2
Summary Demographic Data for Enrolled Subjects in Study¹

| Characteristic | Variable | Value |
|---|---|---|
| Age (yr) | mean | 31 |
| | range | 18-44 |
| Race | Caucasian, n (%) | 3 (6.8%) |
| | Hispanic, n (%) | 41 (93.2%) |
| Gender | Male, n (%) | 25 (56.8%) |
| | Female, n (%) | 19 (43.2%) |
| Weight (kg) | mean | 69.5 |
| | range | 55.3-87.9 |
| Height (cm) | mean | 165 |
| | range | 153-180 |

¹N = 44

All subjects were randomly assigned in a 1:1 ratio to Group A or Group B. Subjects in Group A received a twice daily dose of 500 mg Metformin (Glucophage) for 3.5 days in Period 1. After a washout period of 3 days, these subjects received a daily dose of 60 mg Trospium (Sanctura XR®) for 10 days in Period 2, followed by combined dosing of a twice daily dose of 500 mg Metformin for 3.5 days and a daily dose of 60 mg Trospium for 4 days. Subjects in Group B received a daily dose of 60 mg Trospium for 10 days in Period 1, followed by combined dosing of a daily dose of 60 mg Trospium for 4 days and a twice daily dose of 500 mg Metformin for 3.5 days. After a washout period of 3 days, these subjects received a twice daily dose of 500 mg Metformin for 3.5 days in Period 2.

For plasma sampling at each pharmacokinetics time point, approximately 10 mL of blood was drawn into a green-top sodium heparin tube, and inverted at least 10 times to insure adequate mixing of blood and anticoagulant. The green-top tube was placed in an ice bath for a minimum of 5 minutes before being centrifuged for 10 minutes at approximately 2,000 g at 4° C. Immediately after centrifugation, the plasma samples were transferred into 2 separate duplicate labeled polypropylene cryovials (minimum of 1 mL plasma in each). Plasma samples were kept frozen at all times (−20° C. or below) until bioanalysis.

For urine sampling at each pharmacokinetics time point, baseline urine samples were collected at approximately 30 minutes prior to dosing, and all urine samples were collected at each time interval with instructions to the subjects to completely empty their bladder at the end of every collection period. The collected urine samples were mixed thoroughly by shaking or stirring the container or by carefully pouring the urine back and forth between two containers several times. The total volume of the urine samples were recorded and two 10 mL were stored at −20° C. or below in polypropylene tubes until bioanalysis.

The concentrations of trospium in plasma and urine were measured using a validated liquid chromatography tandem mass spectrometry method (LC-MS/MS). All bioanalytical methods showed acceptable precision and accuracy for the quality control samples, and all plasma and urine samples were analyzed within pre-determined stability window.

LC-MS/MS of trospium in plasma involved the addition of 60 pg of clidinium to 0.2 mL of plasma sample. Solid phase extraction cartridges were preconditioned with 1 mL of methanol, followed by 1 mL of 50 mM ammonium acetate at pH 9.0. Samples were transferred to the cartridges and were allowed to pass through with positive pressure. Columns were washed three times with water followed by a single wash with methanol. The compounds of interest were eluted with 1.00 mL of 0.1% HCl in methanol. The extracts were evaporated to dryness under a stream of nitrogen at approximately 35° C. The dried residues were reconstituted in 50:50 methanol:water (200 μL), vortexed and centrifuged. The samples were transferred to autosampler vials and ten to twenty microliters were injected onto the LC-MS/MS system. LC-MS/MS of trospium in urine involved the addition of 15 ng of clidinium to 0.05 mL of urine sample. Samples were vortexed and a 50 μL aliquot of each was transferred to a clean tube. Each sample was then diluted with 950 μL of 1:1 methanol:water. The samples were vortexed and 200 μL were transferred to autosampler vials. Five to ten microliters were injected onto the LC-MS/MS system. The concentration of trospium in plasma and urine samples were measured using a validated LC-MS/MS method with a lower limit of quantitation (LLOQ) of 0.04 ng/mL and 25 ng/mL respectively.

LC-MS/MS of metformin in plasma involved the addition of 0.2 mL of internal standard (2500 ng/mL metformin-d6) to 0.1 mL of plasma sample. A 0.400 mL aliquot of the protein precipitation reagent (acetonitrile) was added to the sample. The samples were mixed, centrifuged, and a portion of the supernatant was transferred to a clean 10-mL conical glass tube. The samples were dried under nitrogen in a Turbovap at approximately 40° C. The dried residues were reconstituted in acetonitrile, vortex mixed, and centrifuged. A 5 μL aliquot was injected onto the LC-MS/MS system. LC-MS/MS of metformin in urine involved the addition of 1.45 mL of water and 0.0250 mL of internal standard (15.0 pg/mL Metformin-d6) to 0.025 mL of urine sample. Samples were vortexed and a 0.05 mL aliquot of the diluted sample was transferred to 1.5-mL microcentrifuge tubes. A 0.400 mL aliquot of acetonitrile (protein precipitation reagent) was added to all tubes. The samples were vortex mixed, centrifuged, and a portion of the supernatant was transferred to appropriately labeled autosampler vials. A 5 μL aliquot was injected onto the LC-MS/MS system. The concentration of metformin in plasma and urine samples were measured using a validated LC-MS/MS method with a LLOQ of 10 ng/mL and 1 pg/mL respectively.

Mean and individual concentration data of metformin and trospium are listed in Tables 3 and 4.

TABLE 3
Steady-State Plasma Concentrations of Metformin¹

| | | Glucophage ®/ | |
|---|---|---|---|
| Glucophage ® | | Sanctura XR ® | |
| Dosage Regime (N = 43)¹ | | Dosage Regime (N = 43)¹ | |
| Time | Metformin Concentration | | Metformin Concentration |
| (hr) | (ng/mL) | Time (hr) | (ng/mL) |
| 0 | 119 (68.7%) | 0 | 117 (46.0%) |
| 0.5 | 582 (43.3%) | 1 | 714 (38.1%) |
| 0.75 | 659 (36.8%) | 2 | 679 (32.1%) |
| 1 | 691 (37.3%) | 3 | 625 (33.1%) |
| 2 | 638 (36.8%) | 4 | 592 (28.5%) |
| 3 | 583 (37.9%) | — | — |
| 4 | 534 (38.9%) | — | — |
| 6 | 350 (39.3%) | 5 | 539 (27.6%) |
| 8 | 230 (40.1%) | 7 | 334 (37.6%) |
| 10 | 158 (39.3%) | 9 | 226 (37.7%) |
| 12 | 109 (37.7%) | 11 | 155 (43.6%) |
| 14 | 80.3 (39.1%) | — | — |
| 16 | 62.2 (38.6%) | 15 | 83.0 (42.5%) |
| 24 | 33.5 (45.8%) | 23 | 38.7 (52.4%) |

¹Subject 14 was discontinued by the investigator at Day 13 due to flu-like symptoms.

TABLE 4
Steady-State Plasma Concentrations of Trospium

| | | Sanctura XR ®/ | |
|---|---|---|---|
| Sanctura XR ® | | Glucophage ® | |
| Dosage Regime (N = 44) | | Dosage Regime (N = 43)¹ | |
| Time | Trospium Concentration | | Trospium Concentration |
| (hr) | (pg/mL) | Time (hr) | (pg/mL) |
| 0 | 676 (55.4%) | 0 | 455 (67.0%) |
| 1 | 831 (51.4%) | 1 | 618 (54.3%) |
| 2 | 1360 (81.1%) | 2 | 897 (46.8%) |
| 3 | 1570 (76.9%) | 3 | 1050 (62.4%) |
| 4 | 1540 (74.6%) | 4 | 1020 (55.1%) |
| 5 | 1530 (81.2%) | 5 | 979 (56.0%) |
| 6 | 1660 (83.1%) | 6 | 1010 (63.5%) |
| 8 | 1120 (76.9%) | 8 | 739 (75.9%) |
| 10 | 878 (70.7%) | 10 | 611 (64.2%) |
| 12 | 781 (64.3%) | 12 | 564 (77.9%) |
| 16 | 676 (59.5%) | 16 | 509 (73.0%) |
| 24 | 655 (50.3%) | 24 | 454 (59.7%) |

¹Subject 14 was discontinued by the investigator at Day 13 due to flu-like symptoms.

A model-independent approach was used to calculate the plasma and urine pharmacokinetic parameters of trospium and metformin for each subject including peak plasma concentration of metformin (ng/mL) and trospium (ng/mL) (Cₘₐₓ) and time to peak plasma concentration (Tₘₐₓ). The half-life of drug (T_{1/2}) was calculated as 0.693/Ke, where Ke was estimated by linear regression of logarithmic transformed concentration versus time. A minimum number of three data points were used in calculating Ke. Only the data points which were judged to describe the terminal log-linear decline were used in the regression. Half-life values were not determined if there were not enough data points to describe log-linear decline.

The steady state area under the plasma concentration-time curve within dosing interval was calculated by the linear trapezoidal rule (AUC₀₋₁₂ for metformin, AUC₀₋₂₄ for trospium). Oral clearance was calculated as dose divided by AUC₀₋₁₂ for metformin and as dose divided by AUC₀₋₂₄ for trospium (CLₛₛ/F).

Pharmacokinetic drug-drug interaction was evaluated by calculating the 90% confidence interval (CI) of the ratio of estimated geometric means for AUC₀₋₁₂ (metformin), AUC₀₋₂₄ (trospium), and Cmax values obtained following alone and combination treatments. The alone treatment was used as the reference for each respective drug.

The cumulative amount of drug excreted unchanged in the urine during the 12-hour or 24-hour dosing interval (A_{e,0-12} for metformin, A_{e,0-24} for trospium) was calculated for each subject. The percentage of dose excreted in urine was determined from the ratio of cumulative amount of drug excreted in the urine to the administered dose. Renal clearance (CL_{R}) was calculated by dividing the cumulative amount of drug excreted in the urine during the dosing interval divided by the plasma AUC of the corresponding dosing interval of each drug. A paired t-test was performed to compare these parameters between the alone and combination treatments of each compound at the significance level of 0.05.

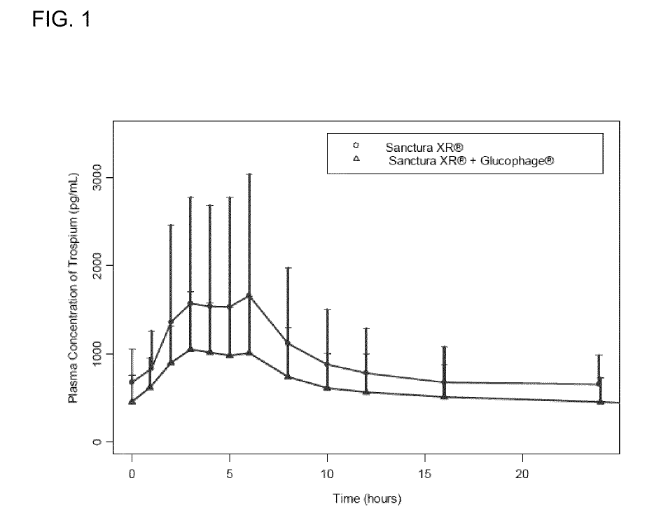

Following twice daily administration of 500 mg Metformin to healthy subjects for 3.5 days, plasma concentration of Metformin peaked at approximately 1.39 hours post-dose, and declined with a mean half life of 7.14 hours (Table 4). The steady-state Cₘₐₓ and AUC₀₋₁₂ values of metformin were 739 ng/mL and 4450 ng·hr/mL, respectively (Table 4). Mean and individual concentration-time profiles of metformin are provided in FIG. 1.

Co-administration of Trospium did not alter the steady-state pharmacokinetics of Metformin. Plasma concentration of Metformin peaked at approximately 1.82 hours post-dose and the mean half life was 5.73 hours (Table 5). Based on the steady-state Cₘₐₓ and AUC₀₋₁₂ values, the combination treatment of Metformin and Trospium was bioequivalent to the Metformin alone treatment; the 90% confidence intervals were 96.7-109% for Cₘₐₓ and 105-120% for AUC₀₋₁₂ (Table 5). The percentage of dose excreted in urine was increased by approximately 13% (p value<0.05), and there was no statistically significant changes in renal clearance of metformin when the drugs were coadministered (p value=0.407) (Table 5).

TABLE 5
Steady-State Plasma and Urine Pharmacokinetic Parameters
of Metformin¹

| Metformin | | Glucophage ®/ |
|---|---|---|
| Pharmacokinetic | Glucophage ® | Sanctura XR ® |
| Parameter | Dosage Regime | Dosage Regime |
| N | 43¹ | 43¹ |
| Cₘₐₓ (ng/mL) | 739 ± 263 | 753 ± 252 |
| Tₘₐₓ (hr) | 1.39 ± 1.04 | 1.82 ± 1.10 |
| AUC₀₋₁₂ (ng · hr/mL) | 4450 ± 1590 | 4900 ± 1550 |
| T_{1/2} (hr) | 7.14 ± 3.37 | 5.73 ± 1.62 |
| CLₛₛ/F (L/hr) | 127 ± 46 | 110 ± 27 |
| A_{e,0-12} (mg) | 141 ± 50 | 160 ± 44 |
| A_{e,0-12}/Dose (%) | 28.3 ± 10.0 | 31.9 ± 8.8 |
| CL_{R} (L/hr) | 33.0 ± 8.5 | 33.7 ± 8.5 |

| | |
|---|---|
| Percentage Ratio and | 103 (96.7-109) |
| 90% CI of the Geometric | |
| Mean of Cₘₐₓ | |
| (Combination/Alone) | |
| Percentage Ratio and | 112 (105-120) |
| 90% CI of the Geometric | |
| Mean of AUC₀₋₁₂ | |
| (Combination/Alone) | |

¹Subject 14 was discontinued by the investigator at Day 13 due to flu-like symptoms.
Lower limit of quantitation for metformin in plasma = 10 ng/mL
Lower limit of quantitation for metformin in urine = 1 μg/mL

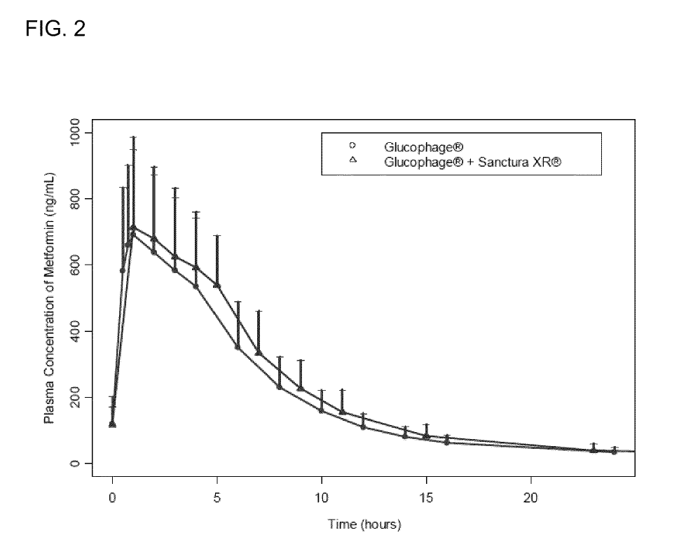

Following daily administration of 60 mg Trospium to healthy subjects for 10 days, plasma concentration of Trospium peaked at approximately 4.42 hours post-dose (Table 5). The steady-state Cₘₐₓ and AUC₀₋₂₄ values of Trospium were 1.87 ng/mL and 22.7 ng·hr/mL, respectively (Table 5). Mean and individual concentration-time profiles of trospium are provided in FIG. 2.

Co-administration of Metformin did not alter the absorption rate of Trospium, with Tₘₐₓ also occurring at approximately 4 hours postdose. Metformin reduced the steady-state Cₘₐₓ and AUC₀₋₂₄ values of Trospium by approximately 34% and 29%, respectively (Table 6). The percentage of dose excreted in urine was reduced by 30% (p value<0.0001), and there was no statistically significant changes in renal clearance of Trospium when the drugs were co-administered (p value=0.880) (Table 6).

TABLE 6
Steady-State Plasma and Urine Pharmacokinetic Parameters
of Trospium

| Trospium | | Glucophage ®/ |
|---|---|---|
| Pharmacokinetic | Glucophage ® | Sanctura XR ® |
| Parameter | Dosage Regime | Dosage Regime |
| N | 44 | 43¹ |
| Cₘₐₓ (ng/mL) | 1.87 ± 1.44 | 1.17 ± 0.71 |
| Tₘₐₓ (hr) | 4.42 ± 1.94 | 4.12 ± 1.60 |
| AUC₀₋₂₄ (ng · hr/mL) | 22.7 ± 14.4 | 15.6 ± 9.5 |
| T1/2 (hr) | | |
| CLₛₛ/F (L/hr) | 3510 ± 1920 | 4830 ± 2320 |
| A_{e,0-24} (mg) | 0.400 ± 0.247 | 0.281 ± 0.156 |
| A_{e,0-24}/Dose (%) | 0.667 ± 0.411 | 0.468 ± 0.260 |
| CL_{R} (L/hr) | 18.4 ± 4.8 | 18.4 ± 5.4 |

| | |
|---|---|
| Percentage Ratio and | 66.3 (61.0-72.0) |
| 90% CI of the Geometric | |
| Mean of Cₘₐₓ | |
| (Combination/Alone) | |
| Percentage Ratio and | 70.8 (66.2-75.7) |
| 90% CI of the Geometric | |
| Mean of AUC₀₋₂₄ | |
| (Combination/Alone) | |

¹Subject 14 was discontinued by the investigator at Day 13 due to flu-like symptoms.
Lower limit of quantitation for trospium in plasma = 0.04 ng/mL
Lower limit of quantitation for trospium in urine = 25 ng/mL

A drug-drug interaction study was conducted to evaluate the pharmacokinetics of Trospium and Metformin when 60 mg Trospium was coadministered with 500 mg Metformin under steady state conditions in 44 healthy subjects. The steady-state pharmacokinetics of Metformin was not affected by the concomitant use of Trospium. Co-administration of Metformin reduced the steady-state systemic exposure of Trospium by approximately 34% for Cₘₐₓ and by 29% for AUC₀₋₂₄. The renal clearance of Trospium remained unchanged when the drugs were co-administered.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A composition comprising a Trospium and a Metformin.

2. The composition of claim 1 wherein the composition includes at least 60 mg of Trospium.

3. The composition of claim 1 wherein the composition includes at least 500 mg of Metformin.

4. The composition of claim 1 wherein the composition contains at least 60 mg of Trospium and at least 500 mg of Metformin.

5. The composition of claim 1 wherein the composition contains at least 40 mg of Trospium and at least 400 mg of Metformin.

6. The composition of claim 1 wherein the Trospium is Trospium chloride.

7. The composition of claim 1 wherein the composition is formulated as an extended release delivery platform.

8. The composition of claim 1 wherein the composition is formulated as an extended release delivery platform with a delayed release component.

9. The composition of claim 1 wherein the extended release delivery platform comprises a pH dependent release.

10. The composition of claim 1 wherein the extended release delivery platform comprises a time dependent release.

11. The composition of claim 1 wherein the composition is administered orally.

12. The composition of claim 1 wherein the composition is administered once daily.

13. A method of treating an overactive bladder disorder, the method comprising the step of administering the composition of claim 1 to an individual suffering from overactive bladder, wherein administration reduces a symptom associated with the overactive bladder disorder.
